(19) United States
(12) Patent Application Publication
(10) Pub. No.: US 2020/0237764 A1
(43) Pub. Date: Jul. 30, 2020

(54) DRUG COMBINATIONS FOR TARGETING MULTIPLE MUTATIONS IN CANCER

(71) Applicant: CureMatch, Inc., San Diego, CA (US)

(72) Inventors: Igor Flint Tsigelny, San, CA (US); Amélie Clémence Boichard, ECKBOLSHEIM (FR); Kevin Toivo Busch, San Diego, CA (US)

(73) Assignee: CureMatch, Inc., San Diego, CA (US)

(21) Appl. No.: 16/638,122

(22) PCT Filed: Aug. 13, 2018
(86) PCT No.: PCT/US18/46570

§ 371 Date: Feb. 10, 2020

Related U.S. Application Data
(60) Provisional application No. 62/544,693, filed on Aug. 11, 2017.

(51) Int. Cl. A61K 31/519; C12Q 1/6886; G01N 33/50; A61K 31/44; A61P 35/00

(52) U.S. Cl. CPC A61K 31/519; C12Q 1/6886; A61P 35/00; A61K 31/44; G01N 33/5011

(57) ABSTRACT

Disclosed are methods for treating a cancer in a patient. The method comprises: (a) defining a set of substances targeting pathogenic genes identified by screening of a sample of cancer cells from a patient by NGS or other technique, (b) identifying two or more target genes in the cancer cells, each of which containing an actionable mutation and (c) testing, using in vitro culture cell experiments, the efficacy of one or more substances (administered sequentially or concurrently) targeting the actionable mutation for each of the two or more target genes identified and (d) designating potential efficacious therapeutic options that will be used to treat the patient's cancer.

### CROSS-REFERENCE TO RELATED APPLICATIONS

The present application is a continuation of International Application No. PCT/US18/46570 entitled “Drug Combinations for Targeting Multiple Mutations in Cancer” filed on Aug. 13, 2018, which published in English as WO 2019/033123 on Feb. 14, 2019, which claims priority from U.S. Provisional Application Ser. No. 62/544,693 entitled “Drug Combinations for Targeting Multiple Mutations in Cancer” filed on Aug. 11, 2017, which are each expressly incorporated herein by reference in its entirety.

### STATEMENT REGARDING FEDERALLY SPONSORED RESEARCH OR DEVELOPMENT

Not Applicable.

### FIELD

The present invention is related to cancer treatment and, more particularly, to the identification and concurrent targeting of multiple cancer cell mutations.

### INTRODUCTION

With the development of advanced molecular techniques, personalized medicine has emerged to the forefront in cancer diagnosis and treatment (1). This has resulted in a shift from cytotoxic, non-specific chemotherapies to molecularly targeted approaches (2). Such targeted approaches have largely been possible as a result of the development of next-generation sequencing (NGS) techniques which perform high-throughput, massive parallel sequencing (3, 4).

Pancreatic ductal adenocarcinoma (PDAC) is an exocrine pancreatic tumor that develops from cells lining the small tubes or ducts in the pancreas: It is an extremely aggressive cancer in that PDAC accounts for up to 4% of all cancer related deaths world-wide with a 5-year survival rate of only about 25% (5).

### SUMMARY

Accordingly, the inventors herein have succeeded in devising an improved approach for the treatment of cancer including PDAC. The approach involves targeting actionable mutations in two or more genes or pathways in the cancer cells.

Thus, in various embodiments, the present invention is directed to methods for treating a cancer in a patient in need thereof. The method includes identifying two or more target genes in the cancer cells (using NGS sequencing or other methods) each of which has a pathogenic and actionable mutation; (a) a list of gene-drug substances interactions, and (b) administering to the patient one or more substances targeting the actionable mutation for each of the two or more target genes identified in the patient's tumor/cancer sample. In various aspects, the sample may be cancer cells or a cell-free sample containing cancer DNA and the two or more target genes map to different pathways. In various embodiments the cancer may be PDAC and the two or more target genes may be KRAS (or genes which signal through the mitogen/extracellular signal-related kinase (MEK) pathway) and ABL1 (or genes which signal through the tyrosine kinase (TK) pathway). The inhibitors may be the mitogen/extracellular signal-related kinase (MEK) inhibitor, trametinib and the multiple tyrosine kinase inhibitor, regorafenib).

In various other embodiments are directed to methods for identifying a treatment for a patient having cancer. The method may include (a) obtaining from the patient, a sample comprising cancer cells (b) screening the sample using an NGS technique, (c) identifying two or more target genes in the cancer cells each of which has a pathogenic and actionable mutation, (d) culturing the cancer cells in presence of one or more substances targeting the actionable mutation for each of the two or more identified target genes identified in (c), (e) measuring cancer cell viability in presence of the one or more substances and (f) determining if the viability of the cells in the presence of the one or more targeted substance is (i) less than the viability in absence of the two or more substances; (ii) less than the viability in the presence of one or more standard-of-care, non-targeted, substance; (iii) less than the viability in the presence of a targeted but non-matched substance (negative controls). In various aspects, each of the two or more identified target genes affects a different pathway. In various embodiments, the cancer is PDAC and the two or more identified target genes are KRAS and ABL1. The inhibitors may be the mitogen/extracellular signal-related kinase (MEK) inhibitor, trametinib and the tyrosine kinase inhibitor, regorafenib. The standard-of-care, non-targeted, substance may be gemcitabine and the targeted but non-matched substance may be palbociclib.

Various other embodiments are directed to a kit for identifying a treatment for a patient having cancer. The kit includes: (a) a list of patient's cancer cells aberrations obtained by NGS (or other technique); (b) a list of gene-drug substances interactions; (c) one or more substances targeting the actionable mutation for each of the two or more target genes identified from the list (a); and (d) a medium for culturing cancer cells from the patient in presence of each of the one or more substances in (b), packaged in one or more containers. In various embodiments, the cancer is PDAC and the two or more identified target genes are KRAS and ABL1. The inhibitors may be the mitogen/extracellular signal-related kinase (MEK) inhibitor, trametinib and the tyrosine kinase inhibitor, regorafenib.

These and other features, aspects and advantages of the present teachings will become better understood with reference to the following description, examples and appended claims.

### DRAWINGS

Those of skill in the art will understand that the drawings, described below, are for illustrative purposes only. The drawings are not intended to limit the scope of the present teachings in any way.

FIG. 1. This figure illustrates the effects of monotherapies on CAPAN2 cell survival showing graphs of CAPAN2 cells treated with increasing concentrations of either (A) gemcitabine, (B) trametinib, (C) regorafenib, or (D) palbociclib in which the black circles (lower curve in each graph) is the drug treatment and the red (in color version) circles (upper curve in each graph) are DMSO-treated at concentrations equivalent to the percentage of DMSO in the serial dilutions of the drug wherein, after 48 hours of exposure to the drugs, concentrations as high as 1 mM for gemcitabine (B) have little effect on inducing cell death, while both matched monotherapies, trametinib (B) and regorafenib (C) induce significant adverse effects on cell survival and wherein, (D) palbociclib was found to little, if any effect on cell survival, even at higher concentrations. Concentrations of the drugs are shown as the log 10 of the μM concentration (e.g., 1000 μM=3.00) (Mean±SD; N≥3).

FIG. 2. This figure illustrates the effects of matched combination therapy. (A) Graph of survival of CAPAN2 cells treated for 48 hrs with serially increasing concentrations of either Regorafenib (blue (in color version) circles), Trametinib (red (in color version) triangles), or a 1:1 combination of Regorafenib and Trametinib (purple (in color version) squares)—Black upper line is DMSO-treatment wherein the graph of cell survival following treatment with the 1:1 combination of the two drugs revealed a biphasic type of curve with two distinct areas of significantly decreased cell survival surrounding an area of increased cell survival and wherein the highlighted area (shaded box) between the dashed boxes indicates area of potential hormesis (B-C) wherein each set of three bars depicts Regorafenib on the left, Trametinib in the middle, and Regorafenib and Trametinib on the right, and cell death in the highlighted areas (surrounded by dashed boxes) are shown in A.

FIG. 3. This figure illustrates the effects of matched combination therapy showing (A) graph of survival of CAPAN2 cells treated for 48 hrs with serially increasing concentrations of either Regorafenib (blue (in color version) circles), Trametinib (red (in color version) triangles), or a 2:1 combination of Regorafenib and Trametinib (purple (in color version) squares)—Black line is DMSO-treatment wherein the graph of cell survival following treatment with the 2:1 combination of the two drugs revealed a biphasic type of curve with two distinct areas of significantly decreased cell survival surrounding an area of increased cell survival. The highlighted area (shaded box) between the dashed boxes indicates area of potential hormesis. (B-C) wherein each set of three bars depicts Regorafenib on the left, Trametinib in the middle, and Regorafenib and Trametinib on the right, and cell death in the highlighted areas (surrounded by dashed boxes) are shown in A.

FIG. 4. This figure illustrates the synergistic effects between Regorafenib and Trametinib in CAPAN2 cells—Combination index (CI) analysis showing CI values generated for the different ratios of Regorafenib to Trametinib. Trendlines indicate CI values at any given effect, and symbols represent CI values derived from actual data points. CI=1, additivity; CI>1, antagonism; CI, <1, synergy. 1:1 Regorafenib:Trametinib curve (blue in color version) has the highest value at EC60, 10:1 Regorafenib:Trametinib curve (green in color version) has the second-highest value at EC60, 5:1 Regorafenib:Trametinib curve (red in color version) has the third-highest value at EC60, and 2:1 Regorafenib:Trametinib curve (yellow in color version) has the fourth-highest value at EC60.

FIG. 5. This figure illustrates the synergistic effects between Regorafenib and Trametinib in CAPAN2 cells—Isobolographic Analysis. (A-B) Isobolograms showing the synergistic effects of Regorafenib and Trametinib at 1:1 and 2:1 combination ratios wherein the diagonal, colored lines indicate additivity, and the colored symbols show dose requirements to achieve 20% (ED80—blue (in color version) lower line), 25% (ED75—yellow (in color version) middle line), or 40% (ED60-red (in color version) upper line) CAPAN2 cell death, respectively and wherein data points below the line of additivity indicate synergy, data points above show denote antagonism.

FIG. 6. Overview of normal KRAS intercellular signaling. Upon binding of a growth factor (triangle labeled growth factor; red/pink in color version) to the extracellular portion of its receptor (Y-shaped structure crossing through plasma membrane labeled growth factor receptor; black and blue in color version) downstream signaling events are initiated through a series of small molecule intermediates (circled (green in color version)) leading to activation of RAS (KRAS, NRAS, or HRAS) which then activates RAF (BRAF, CRAF) which subsequently activates MEK1/2 and ERK1/2 leading to increases in transcription and cellular survival and proliferation.

FIG. 7. Overview of normal ABL signaling (B). ABL is modulated by a number of stimuli, including growth factors (triangle; purple in color version), chemokines (square; blue in color version) and integrin signaling (black column). ABL (circles; orange in color version) is found intercellularly in the cytoplasm (both free and actin-bound) and the nucleus. Nuclear ABL regulates transcription, while cytosolic ABL can be found both free and actin-bound. Free cytosolic ABL has kinase activity and plays roles in cellular chemotaxis and mitogenesis. Actin-bound ABL does not have kinase activity, but can be released from actin in response to integrin signaling.

### DETAILED DESCRIPTION

The present invention is directed to the identification and targeting of two or more mutations in cancer cells in treating cancer patients.

As used herein, the following terms are defined with the following meanings, unless explicitly stated otherwise.

The term “about” when used before a numerical designation, e.g., pH, temperature, amount, concentration, and molecular weight, including range, indicates approximations which may vary by ±5%, ±1% or ±0.1%.

As used in the specification and claims, the singular form “a”, “an” and “the” include plural references unless the context clearly dictates otherwise. For example, the term “a pharmaceutically acceptable carrier” may include a plurality of pharmaceutically acceptable carriers, including mixtures thereof.

The term “and/or” is intended to mean either or both of two components of the invention.

The terms “subject,” “individual” and “patient” are used interchangeably herein and refer to a mammal and, particularly, to a human.

The term “device,” as used herein, refers to an apparatus or system capable of delivering a drug to patient in need thereof.

The term “in need of treatment” and the term “in need thereof” when referring to treatment are used interchangeably and refer to a judgment made by a caregiver, e.g.

physician, nurse, nurse practitioner, that a patient will benefit from treatment.

The term “pharmaceutically acceptable,” as used herein, refers to a component of a pharmaceutical composition that is compatible with the other ingredients of the formulation and not overly deleterious to the recipient thereof.

The term “carrier” refers to a diluent, adjuvant, excipient, or vehicle with which the therapeutic is administered and includes, but is not limited to such liquids and powders that are hydrophilic substances, hydrophobic substances and substances that possess both hydrophilic and hydrophobic properties such as emulsifiers.

The term “therapeutically effective amount,” as used herein, refers to the amount of an active compound or pharmaceutical agent that elicits the biological or medicinal response in a tissue, system, or individual that is being sought by a researcher, healthcare provider or individual.

The term “w/w” as used herein, is intended to refer to mass fraction, i.e., the mass of a component divided by total mass of the whole. The term “% w/w” is intended to refer to the mass fraction multiplied by 100. Similarly, the term “w/v” refers to volume concentration, i.e., the mass of a component divided by total volume of the whole and the term “% w/v” refers to the volume concentration multiplied by 100.

Various embodiment of the present invention are directed to methods for treating a cancer in a patient in need thereof and to methods for identifying a treatment method for a patient having cancer

The term “cancer” refers to a group of diseases in which abnormal cells divide without control often invading nearby tissues and spreading to other parts of the body through the blood and lymph systems. One particular cancer is pancreatic ductal adenocarcinoma (PDAC). PDAC is an extremely aggressive cancer developing from the pancreatic ducts and accounting for up to 4% of all cancer related deaths world-wide with a 5-year survival rate of only about 25% (5).

The term “sample” as used herein, refers to cancerous tissue or group of cells from a patient's cancer such as, for example, from an excisional or incisional biopsy including a core biopsy, needle aspiration biopsy and the like. A “sample” may also be cell-free fluid obtained from the patient containing DNA from the cancer.

A sample from a patient may be screened using next-generation sequencing (NGS). NGS refers to technologies capable of massively parallel sequencing millions of DNA templates (3, 4). The term includes second-generation and third-generation sequencing as distinguished from the first-generation dideoxy ‘Sanger’ sequencing. NGS techniques employ clonal amplification of DNA templates on a solid support matrix followed by cyclic sequencing. Examples of NGS include sequencing-by-synthesis (reversible terminator-based) in such products as MiSeq®, HiSeq® and NextSeq® (Illumina Inc., San Diego, Calif.), Sequencing-by-Synthesis (Semiconductor-based) in such products as Ion Torrent®, Ion Proton®, (Life Technologies® Thermo Fisher Scientific, Waltham, Mass.) and Single molecule real-time sequencing in such a product as PACBIO® RSII (Pacific Biosciences, Menlo Park, Calif.).

The term “pathogenic mutations” refers to genetic alteration that increases an individual's susceptibility or predisposition to a certain disease or disorder, such as cancer.

The term “actionable mutations” refers to mutations that affect genes or pathways that are targetable by drugs in effectively treating a certain disease or disorder, such as cancer (6, 7). The actionable mutations may map to a known pathway such that pathway-targeted therapeutics may be effective (8). Such therapeutics may include inhibitors such as the MEK inhibitor, trametinib and the multiple tyrosine kinase inhibitor, regorafenib, in particular, for the treatment of PDCA.

A proto-oncogene is a normal gene that, when activated by mutation or increased copy number, becomes an oncogene and that can contribute to cancer. Proto-oncogenes may have many different functions in the cell such as providing signals that lead to cell division or regulating apoptosis.

One of the target genes may be a mutant KRAS gene. The mutated KRAS oncogene contributes to the mitogen-activated (MAP) kinase pathway which controls to cell growth and differentiation (9). KRAS is activated by GTP which produces the successive activation of RAF kinases, MEK kinases and ERK kinases and the ERKs phosphorylate transcription factors leading to cell proliferation (10). One actionable MEK inhibitor is trametinib which suppresses ERK phosphorylation through the RAF-dependent activation of MEK (11). Trametinib may be administered at about 1 to about 3 mg/day P.O. and, in particular, at 2 mg/day P.O.

Another of the target genes may be the ABL1 proto-oncogene which encodes a protein tyrosine kinase which when aberrantly activated to become an oncogene, disturbs downstream signaling pathways, causing enhanced proliferation, differentiation arrest and resistance to cell death (12). One actionable ABL1 kinase inhibitor is regorafenib which is an inhibitor of multiple protein kinases (13). Regorafenib may be administered at about 80 to about 240 mg/day P.O. and, in particular, about 160 mg/day P.O.

Various other embodiments are directed to kits identifying a treatment method for a patient having cancer. The kits may include a list of gene-drug interactions; Sample of cancer cells from the patient. List of aberration in these cancer cells obtained by NGS or other technique. The kit generates a list of possible combination targeted therapies. In kits for identifying a treatment method for a patient having PDAC, the two target genes may be KRAS and ABL1. For these two target genes, trametinib (MEK inhibitor) and regorafenib (tyrosine kinase inhibitor) are included in the kit. Further included in the kit may be media for culturing sample cancer cells obtained from the patient. Such media may be any standard culture media such as, for example, McCoy's Modified 5A media supplemented with 10% FCS, 1X penicillin/streptomycin and 1× amphotericin as may be obtained from Life Technologies-Gibco, Carlsbad, Calif. Each of the components of the kit are packaged along with appropriate instructions in one or more containers.

### EXAMPLES

Aspects of the present teachings may be further understood in light of the following examples, which should not be construed as limiting the scope of the present teachings in any way.

### Example 1

This example illustrates the effectiveness of concurrently/simultaneously targeting multiple actionable mutations in cancer cells using a combination of therapeutic agents in a culture method.

### INTRODUCTION

Cancer treatment is still largely a “one size fits all” approach with the majority of treatment options and procedures (e.g., surgery, radiation therapy, chemotherapy) aimed largely at fighting a particular type of cancer (e.g., liver cancer, lung cancer, colorectal cancer) (14) However, over the past several years, the utilization of next-generation sequencing strategies has not only greatly increased our knowledge of the genetic alterations that drive cancer susceptibility and progression, but it has also clearly illustrated the unique nature of an individual patient's cancer (15). Together with advances in the development of therapies targeting the proteins and pathways affected by many of these genetic alterations, this has raised the possibility of utilizing personalized cancer treatment strategies aimed at attacking one patient's cancer (16).

In support of this notion, a number of studies have demonstrated the efficacy of monotherapies which target a particular mutation in the treatment of several different types of cancer. Although not without some risks (e.g., toxicities and drug resistances), many of these studies have demonstrated significant increases in response rates and progression free survival compared to non-targeted approaches (16-20). However, as whole genome sequencing has demonstrated, cancer genomes are generally characterized by a cocktail of genetic aberrations resulting from an overall genetic instability (i.e., mutational burden), rather than alterations in a single gene (21). Nevertheless, most cancer patients, for whom targeted therapy is implemented, are generally treated with therapies aimed at a single-agent matched aberration (monotherapy). This is despite the fact that, based on the results of such targeted monotherapies, the inventors herein believe that combinations of therapies matched to the entire set of actionable alterations presented by the cancer genomic profile of the patient would likely result in better response.

As a proof-of-principle of the efficacy of matched combination therapy, we have performed in vitro cell-based survival assays using CAPAN2, an established cell line derived from a human/patient pancreatic ductal adenocarcinoma (22). This cell line has been characterized and harbors several mutations, some of which were matched to available FDA-approved drugs that specifically inhibit the pathways affected by the gene aberrations found in the cells (“matched” therapy). Thus, in this study, comparisons in cell viability were made between cells treated with either: 1) the standard treatment for pancreatic ductal carcinoma (i.e. gemcitabine) (23); 2) matched monotherapy with individual drugs/inhibitors targeting selected single/unique signaling pathways altered in the cells; and 3) combination therapy matched to the same selected aberrations.

### Materials and Methods

Materials

Unless otherwise indicated, all chemicals, including gemcitabine, trametinib, palbociclib, and regorafenib were obtained from SelleckChem (Houston, Tex.). McCoy's 5A modified growth media, penicillin-streptomycin, amphotericin, and fetal calf serum were obtained from Life Technologies-Gibco (Carlsbad, Calif.). All plastic-ware, including tissue culture dishes, serological pipettes, pipette tips, and microfuge tubes were from Fisher Scientific (St. Louis, Mo.).

Selection of Cell Line

The cell line was chosen by analysis of the Cancer Cell Line Encyclopedia (CCLE) which provides access to analysis and visualization of DNA copy number, mRNA expression, mutation data and more, for 1,000 cancer cell lines (24). Criteria for the selection of the cell line of interest included: 1) the total number of mutations presented by the cell line should be at least 5, but no more than 10 (to avoid intricacy of additional confounding factors); 2) the number of actionable targets should be at least 2, but no more than 3 (to limit the number of potential drug combinations to test); 3) the mutations could not be significantly overlapping and should affect distinct oncogenic pathways (to avoid redundancies in the drug treatment). From a set of 18 cell lines, we selected the CAPAN2, which originates from a human pancreatic adenocarcinoma primary tumor.

Cell Culture

The human pancreatic cancer cell line CAPAN2, was purchased from the American Type Culture Collection (ATCC; Manassas, Va.). Cells were grown in McCoy's Modified 5A media supplemented with 10% FCS, 1× penicillin/streptomycin and 1× amphotericin. For testing, cells were released from the dishes by treatment with PBS (without calcium) for ˜30 min followed by 0.25% trypsin-EDTA for 5 minutes at 37 C. Cells were collected in a 15 ml centrifuge tube and spun down in a clinical centrifuge for 5 min and then re-suspended in 1 ml of fresh media and the cells were counted using a hemocytometer. Cells (1×10³-5×10³) were then seeded into 96-well plates in 100 μL of media and the cells were incubated for 48 hours. After 48 hours, the media was replaced and the cells were re-incubated for another 24 hours prior to treatment with the drugs.

Cell Treatment, Viability Assay and Dose-Response Assessment

Genomic information (i.e., mutational status, copy number variations, etc) corresponding to the cell line of interest was analyzed and the combination of trametinib (MEKINIST®, MEK inhibitor counteracting the KRAS activating mutation) and regorafenib (STIVARGA®, multi-kinase inhibitor counteracting the ABL1 activating mutation), were selected as a potential therapeutic regimen for CAPAN2 cells since they target these two actionable mutations.

Stock drug solutions were prepared in complete media from master stock solutions prepared in DMSO according to the manufacturer's instructions. Using these stock solutions, 1:1, 2:1, 5:1, and 10:1 volume-volume (v/v) mixtures of regorafenib and trametinib were prepared. These mixtures were then two-fold serially diluted to generate a range of 20 concentrations in each case. The cells were incubated in drug mixtures for 48-72 hours before cell viability assay/assessment.

Cell viability was determined using a WST-1 colorimetric cell proliferation assay (Roche), according to manufacturer's instructions. The stable tetrazolium salt WST-1 is cleaved to a soluble formazan by a complex cellular mechanism that occurs primarily at the cell surface. This reduction is largely dependent on the glycolytic production of NAD(P)H in viable cells. Therefore, the amount of formazan dye formed, and estimated using a spectrophotometer (BIO-TEK 340, BIOTEK) directly correlates to the number of metabolically active cells in the culture. All testing points were done at least in triplicate.

Data were processed in Excel 2016 (Microsoft), GraphPad Prism 5 (GraphPad Software), and Dr Fit (Dr Fit software) (12). The data was used to generate dose-response curves and drug concentrations that exhibited 20%, 25% or 40% of growth inhibition (IC20, IC25 and IC40, respectively) were determined for further analysis.

Isobolographic Analysis

Drugs given in combination may produce effects that are greater than or less than the effect predicted from their individual potencies. Isobolographic analysis, which detects synergy, additivity, or antagonism between a drug pair (26), was carried out to assess the effects of the drug combination. In general, if the drug pair improves the inhibitory potency relative to that of each drug alone, the combination is considered synergistic; if potency remains unchanged, the effect is considered additive; and if potency is reduced, the effect is considered antagonistic. To describe the dose-dependent interaction of trametinib and regorafenib, isobolograms at effect levels of 20%, 25% and 40% inhibition of cancer cell proliferation were created. In each of these, additivity was determined by extrapolating the dose requirements for each drug in combination from its single use (IC20, IC25 and IC40). Data points above or below the line of additivity indicate antagonism or synergy, respectively.

Isobolograms were built by plotting the concentrations of trametinib on the y-axis and the concentration of regorafenib on the x-axis. The isobole of additivity was generated by plotting the IC20 (or IC25, IC40) of each drug (when used in monotherapy) on their respective axis, and connecting them with a diagonal line. The effects of the combination of trametinib and regorafenib at different dose ratios was then determined by plotting their respective IC20s (or IC25, IC40) on this XY graph.

Statistical Analysis

All values were reported as mean+/−SD. The Student's t-test was employed to evaluate the difference between treatments. A p-value lower or equal to 0.05 was considered for significance of all results.

Results

Selection of Cell Line.

The cell line in which to test the efficacy of selected drug regimens was chosen from a database encompassing molecular annotations of −1,000 cell lines (Cancer Cell Line Encyclopedia, CCLE, Novartis/Broad Institute) (24). Using a number of criteria (e.g., number and types of mutations, pathogenicity and actionability of the mutations) the list of cell lines of interest was reduced to 18 possible choices (Table 1).

TABLE 1
Possible Cancer Cell Lines to be Tested

| NAME | Gene Altered | Aberration | Actionable |
|---|---|---|---|
| A-673 or A673 | BRAF | V600E | BRAF |
| | MYH11 | S645L | |
| | NACA | K1759E | |
| | PREX2 | D1019V | |
| ACHN | MYH11 | D1522N | PDE4DIP |
| | PDE4DIP | V486fs | SMO |
| | PREX2 | Q1665fs | |
| | SMO | K635fs | |
| | | N608S | |
| CAL-85-1 | BCL9 | G696A | ERBB4 |
| | ERBB4 | M1017T | TP53 |
| | GTSE1 | P595L | |
| | RUNX2 | P253L | |
| | TP53 | K132E | |
| CAPAN-2 | ABL1 | G1079D | ABL1 |
| | CAD | ? | FANCC |
| | CREB3L2 | T100del | KRAS |
| | FANCC | E521K | |
| | KRAS | G12V | |
| | LRRK2 | N202Y | |
| | MLL3 | Y816fs | |
| | SPTA1 | E1115* | |
| DEL | CREBBP | E1630_splice | CREBBP |
| | LRP1B | S3591Y | PDE4DIP |
| | PDE4DIP | L601fs | RAC1 |
| | RAC1 | Q1665fs | |
| | TP53 | V104M | |
| | | A107E | |
| | | S215C | |
| EHEB | CAD | N154S | |
| | LZTR1 | R810W | |
| | RAD50 | M1I | |
| G-401 | CREB3L2 | T100del | LRRK2 |
| | LRRK2 | ? | |
| | NSD1 | R1188S | |
| G-402 | AFF1 | R546W | NF1 |
| | CREB3L2 | T100del | |
| | MLLT3 | ? | |
| | MSH3 | PPA66del | |
| | NF1 | P1358S | |
| JL-1 | BTG1 | N165S | |
| | DAXX | R311Q | |
| JVM-2 | MAP3K7 | M392K | PDE4DIP |
| | MSH3 | AAAAAAAP | |
| | NCOA2 | P56del | |
| | PDE4DIP | G27R | |
| | | L601fs | |
| | | P63T | |
| | | E243fs | |
| MHH-CALL-2 | NF1 | Q535* | NF1 |
| | PDE4DIP | L601fs | PDE4DIP |
| | TET1 | E243fs | |
| | | F329C | |
| MHH-NB-11 | ARID1A | Q1367_splice | CDKN2C |
| | CARD11 | K986M | HSP90AA1 |
| | CDKN2C | V130A | PDE4DIP |
| | CDX2 | V306M | |
| | CLTCL1 | V1401I | |
| | CREB3L2 | T100del | |
| | HSP90AA1 | S16F | |
| | MSH3 | AAAAAAAP | |
| | PDE4DIP | P56del | |
| | | PPA66del | |
| | | I2090V | |
| MOG-G-CCM | CREB3L2 | T100del | PDE4DIP |
| | PDE4DIP | L601fs | RB1 |
| | RB1 | E243fs | TP53 |
| | TP53 | W563* | |
| | | A159V | |
| NCI-H1755 or | BRAF | G469A | BRAF |
| H1755 | CXCR4 | I226V | CXCR4 |
| | PDE4DIP | Q1665fs | PDE4DIP |
| | TGFBR2 | E150fs | TGFBR2 |
| NCI-H2052 or | PDE4DIP | V486fs | PDE4DIP |
| H2052 | RPTOR | Q1665fs | RPTOR |
| | | R532Q | |
| SW1990 | JAK3 | Q510R | JAK3 |
| | KRAS | G12D | KRAS |
| TC-71 | ERBB4 | A1236V | ERBB4 |
| | LRP1B | C2871* | TP53 |
| | MLL3 | P4726S | |
| | NUP98 | Y816fs | |
| | TP53 | G98R | |
| | | R213* | |
| ZR-75-1 | HRAS | E162K | HRAS |
| | LRP1B | P78T | PTEN |
| | PTEN | L108R | |
| | ZNF521 | D880E | |

From this list, CAPAN2, an epithelial cell line derived from a pancreatic ductal adenocarcinoma (PDAC) of a 56 year-old Caucasian male (23), was chosen for analysis. In optimum culture conditions, the cells present a doubling time of around 96 h (9). According to the CCLE, CAPAN2 cells bear ˜8 missense mutations, of which 3 (KRAS p.G12V; ABL1 p.G1060D; FANCC p.E521K) were found to be actionable targets (Table 1). However, since FANCC p.E521K is a heterozygous mutation for which the functional significance is unclear (27), we focused attention on the 2 other actionable mutations.

KRAS

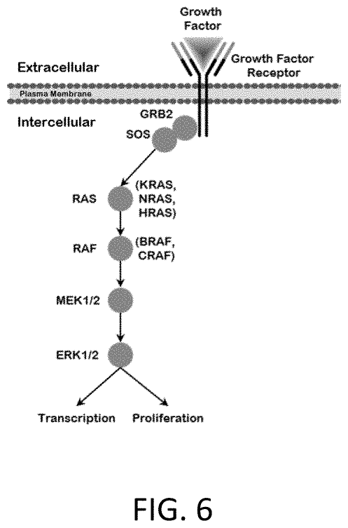

KRAS, a small GTPase, functions in regulating cell growth and proliferation through its participation in the mitogen-activated protein kinase (MAPK) signal transduction pathway (FIG. 6). Under normal conditions, KRAS is activated when a growth factor (e.g. EGF, VEGF, PDGF, etc) binds to its corresponding receptor tyrosine kinase (e.g. EGFR, VEGFR, PDGFR, etc). This inducible activation of KRAS then

stimulates the downstream molecules, RAF (ARAF, BRAF and CRAF), which subsequently phosphorylates and activates the downstream mitogen-activated protein (MAP) kinase kinases, MEK1 and MEK2, and ERK1 and ERK2 (FIG. 6). Ultimately, ERK1/2 translocate to the nucleus and enhance the transcription of genes necessary for the cell proliferation. In the absence of growth factor stimulation, KRAS is normally kept inactivate by dephosphorylation of GTP to GDP. However, in the mutated form, KRAS loses its ability to cleave GTP to GDP and therefore it remains constitutively active (even in the absence of growth factor binding)—leading to uncontrolled continuous cell proliferation and growth.

TABLE 2
List of gene aberrations found in CAPAN2 cells. Altered genes are
listed and their genomic sequence (if known) and resulting protein
sequence are shown. The effect of the mutation on the function of the
protein and whether the aberration is actionable (i.e., is there a
drug to treat directly or indirectly the mutation effect?) are also shown.
CAPAN2 Mutations

| | Gene | Protein | Consequence | |
|---|---|---|---|---|
| Gene | Sequence | Sequence | and Effect | Actionable |
| ABL1 | | G1060D | probably | Yes |
| | | | pathogenic by | |
| | | | gain of function | |
| FANCC | | E521K | Effect unknown | Yes |
| KRAS | | G12V | Pathogenic by | Yes |
| | | | gain of function | |
| CAD | c.637+10T > C | | Effect unknown | No |
| CREB3L2 | | T100del | Loss of function, | No |
| | | | resulting effect | |
| | | | unknown | |
| LRRK2 | | N202Y | Effect unknown | No |
| MLL3 | | Y816fs | Pathogenic by | No |
| (=KMT2C) | | | loss of function | |
| SPTA1 | | E1115* | Loss of function, | No |
| | | | resulting effect | |
| | | | unknown | |

Approximately 90% of all PDACs display activating mutations in KRAS, making it the most frequently mutated onco-protein in PDAC (15). Moreover, mutations at codon 12, such as the substitution p.G12V, account for ˜98% of all KRAS mutations in PDAC (16). The p.G12V mutation results in constitutive activation of the kinase (17) and is observed in additional tumor types, such as colorectal and non-small cell lung adenocarcinomas.

ABL1

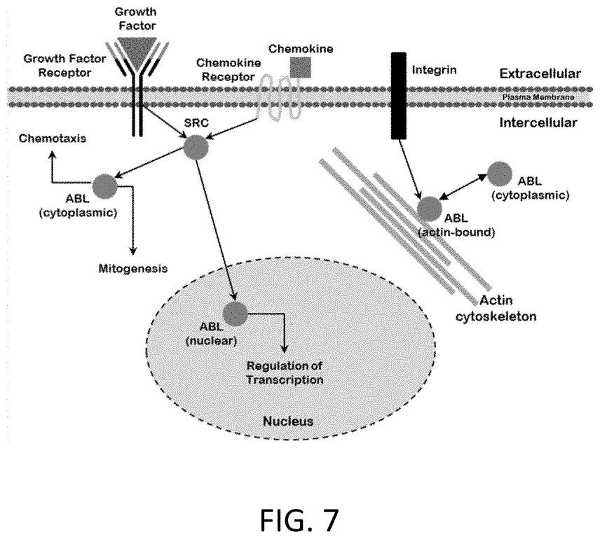

The ABL1 proto-oncogene encodes a non-receptor tyrosine kinase involved in cell differentiation, cell division, cell adhesion and stress response (31) (FIG. 7). ABL1 exhibits a generalized subcellular localization, being found in the nucleus, cytoplasm and bound to the actin cytoskeleton (32). In the nucleus, ABL1 functions in the control of cell-cycle dependent and DNA damage-induced transcription (33). In the cytoplasm, this non-receptor tyrosine kinase is found both free and bound to filamentous actin. As a free molecule, ABL1 is downstream of several potential modulatory signals and regulates, in turn, the activity of a number of downstream proteins involved in cell invasion and growth, while bound to the actin cytoskeleton, this kinase activity is turned off (33) (FIG. 7).

In contrast to the well-established role of the oncogenic fusion protein BCR-ABL1, which is a hallmark of chronic myeloid leukemia leading to the constitutive expression and further hyper-activity of the tyrosine kinase (34), much less is known about the role of ABL1 when mutated by point mutations in solid tumors (31). However, unlike a number of point mutations located within the tyrosine kinase domain of ABL1 which have been found to activate this non-receptor tyrosine kinase leading to cell transformation (35), the p.G1060D mutation of ABL1, seen in these cells, occurs in the actin-binding domain of the kinase. While it has not been functionally characterized, since this domain is a major determinant for the subcellular localization of the kinase to the actin cytoskeleton (which block AB1 kinase activity) and since transforming ABL1 mutations identified to date result almost exclusively in the cytoplasmic accumulation of the kinase (33,34)—this alteration putatively leads to increases in its cytoplasmic level and further activation, since the kinase activity of ABL1 is inhibited when the protein is bound to F-actin (32, 35).

Drug Treatment

Gemcitabine Monotherapy.

Gemcitabine (GEMZAR®) monotherapy, which has been the standard of care for pancreatic cancer for several decades, is the most common cytotoxic drug used in treatment of this disease (23). This pyrimidine analogue is phosphorylated in the cell and gets incorporated into the DNA where it inhibits DNA synthesis (37), therefore targeting all proliferative cells (without restriction to tumor cells), and thus resulting in important side effects (such as severe myelosuppression with neutropenia and bleeding, alopecia, nausea and vomiting, fatigue). Despite the fact that gemcitabine-treatment only results in modest improvements in terms of overall survival when compared to the best supportive care (5 to 6 months, compared to 3 months), as of 2017, gemcitabine remains the standard of care for advanced pancreatic adenocarcinoma (38).

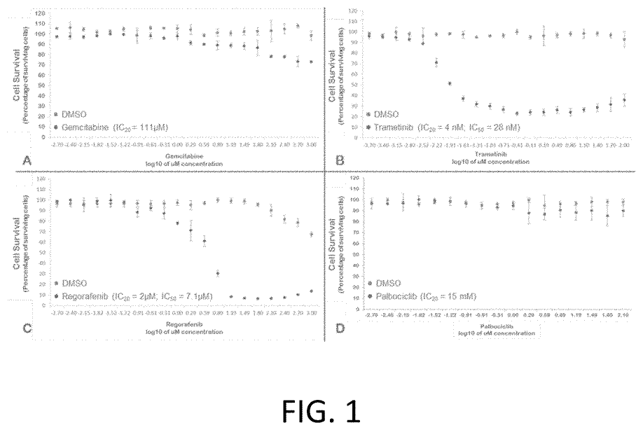

In this study, CAPAN2 cells were treated with a two-fold serial dilution of gemcitabine with concentrations ranging from 2 nM to 1 mM for 48-72 hours. Under these culture conditions, gemcitabine was found to have little, if any, effect on cell survival (FIG. 1A) (IC₂₀=111 μM, with only a maximum decrease in cell viability of −30% achieved using a concentration of 1 mM) (p≤0.001).

Trametinib Monotherapy.

In CAPAN2 cells, the p.G12V mutation in KRAS results in constitutively active mitogen/extracellular signal-related kinase (MEK), which is downstream of KRAS in the MAPK signaling pathway (FIG. 6). Trametinib (MEKINIST®), as a selective inhibitor of MEK is a downstream inhibitor of this constitutively activated pathway (39). In contrast to treatment with gemcitabine, CAPAN2 cells treated with a monotherapy of trametinib in concentrations ranging from 100 μM to 0.2 nM for 48-72 hours significantly decreased cell survival, with an IC₂₀ of 4 nM (p≤0.05) and an IC₅₀ of 28 nM (p≤0.05) (FIG. 1B).

Regorafenib Monotherapy.

As described above, the p.G1060D mutation in ABL1 is likely an activating mutation leading to increases in the cytoplasmic concentration of this non-receptor tyrosine kinase (FIG. 7). Regorafenib (STIVARGA®) is a multi-kinase inhibitor targeting receptor and non-receptor tyrosine kinases, including RET, VEGFR1-3, FGFR1-2, TIE2 and ABL1 among many others (40), and thus should inhibit the activated pathway. Treatment of CAPAN2 cells with a two-fold serial dilution of regorafenib alone (i.e., concentrations ranging from 2 nM to 1 mM) resulted in a significant reduction in cell survival with an IC₂₀ of −2 μM (p≤0.05) (FIG. 1C) and an IC₅₀ of 7.1 μM (p≤0.05).

Palbociclib Monotherapy.

Pancreatic ductal adenocarcinoma has been found to exhibit a range of genetic alterations, including loss or silencing of CDKN2A, a tumor suppressor gene which encodes the p16ink4a protein (an inhibitor of the cyclin dependent kinases 4 and 6 (CDK4/6)) (41). Loss of function mutations of CDKN2A results in deregulation of the cell cycle via CDK4 and CDK6 leading to enhanced cell proliferation. While the status of CDKN2A in CAPAN2 cells remains unclear, some groups have demonstrated the expression of the p16 protein, while others have indicated that CDKN2A is inactivated in these cells (42). The cells were treated for 48-72 hours with a 2-fold serial dilution of the CDK4/6 inhibitor, palbociclib with concentrations ranging from 125 μM to 2 nM. Palbociclib was found to have no significant effect on the survival of the CAPAN2 cells used in this study (FIG. 1D) (IC₂₀=15 mM). This finding demonstrates the persistence of a p16 functional activity and indicates that, at least in this particular strain of CAPAN2 cells, proliferation is not dependent upon CDK4 and/or CDK6. Furthermore, this result also allows palbociclib, as an unmatched monotherapy, to serve as a negative control.

Combination Therapy with Trametinib and Regorafenib.

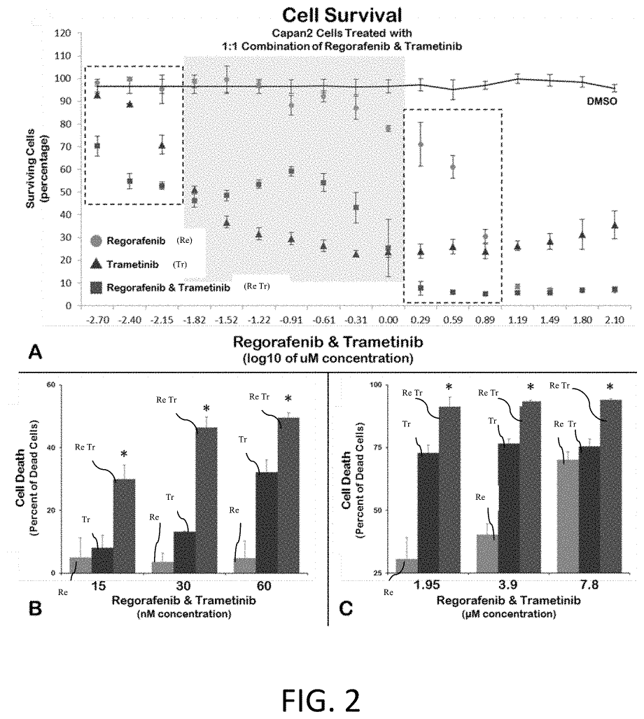

Simultaneous treatment of CAPAN2 cells with trametinib and regorafenib was then used to investigate the effects of matched combination therapies on CAPAN2 cell survival. Co-administration of these two inhibitors at 1:1 concentrations resulted in significant increases in cell death compared to treatment with either drug alone (i.e., monotherapy) with an IC₂₀ of 2 nM (FIG. 2).

Interestingly, however, the dose-response curve of cell viability for this 1:1 combination of these drugs displays a biphasic U-inverted shape, with a loss of efficiency between 15 nM and 1 μM (FIG. 2). Nevertheless, examination of the two regions of concentrations located just before and just after this effect shows statistically significant increases in cell death (FIGS. 2B-2C) (−55% at 300 nM for the 1:1 combination, compared to −2% and −10% for regorafenib and trametinib alone—(p≤0.05)), indicating a potential synergistic inhibitory effect of the combination of drugs on cell proliferation.

To investigate whether the presence of both drugs enhances the individual effects of each drug alone, the “Fixed-Ratio-Model” was employed (43-45). In this model, based on Loewe's concept of (43-45), combination index (CI) values were calculated based on the slope and ICₓ value of each dose-response curve (drug alone or in combination) and used to define whether the drug-drug interactions are synergistic (CI<1), additive (CI=1), or antagonistic (CI>1) (Fi. 4). Following this, the combination index (CI) resulting in a decrease of 20% of cell survival is equal to 0.345, 25% of cell survival is equal to 0.320, while that for 40% survival was found to be greater than 1. This indicates that for ED80 and ED75 there are synergistic effects of the combination of drugs on CAPAN2 cell proliferation, when both drugs are used at equal concentrations. (33).

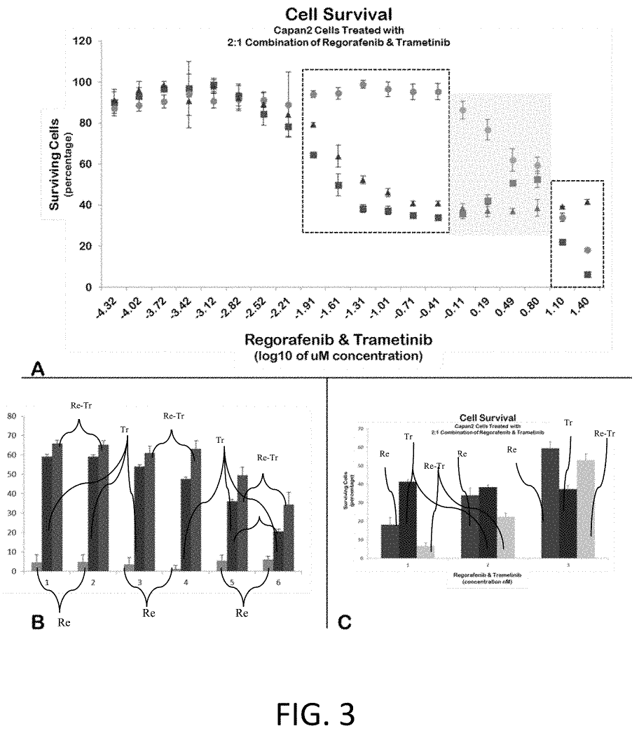

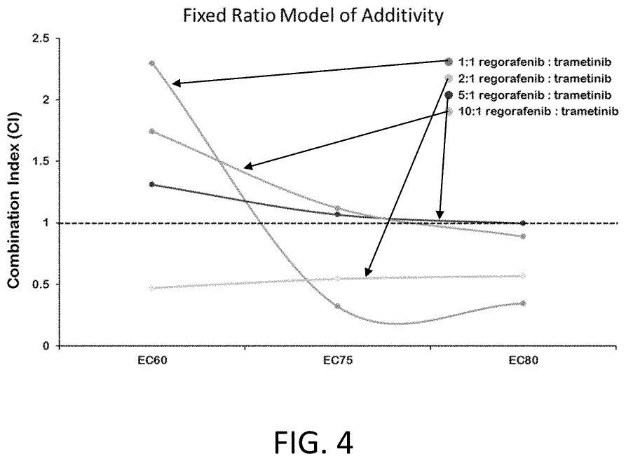

However, co-administration of the drugs at 2:1 concentrations of regorafenib to trametinib was somewhat different (FIG. 3). As above, a biphasic dose response curve was seen, with two areas of increased cell death bordering a small region of apparently increased cell survival, between concentrations 0.78-0.39 μM and 6.25/3.125 μM (FIGS. 4B, 4C). Nevertheless, in both cases (e.g., 1:1, 2:1 concentrations) the overall level of cell survival is significantly less than that seen with regorafenib alone (FIGS. 2-3), and the combination of drugs remains synergistic (CI20%=0.568, CI25%=0.546 and CI40%=0.471 when a ratio 2:1 is used—CI50% was greater than 1) (FIG. 4). Similar experiments were performed at 5:1 and 10:1 concentrations of regorafenib to trametinib, but no significant differences in cell death were seen at these ratios of concentration (data not shown).

Trametinib and Regorafenib Cytotoxicities Synergize in Pancreatic Ductal Adenocarcinoma Cancer Cells.

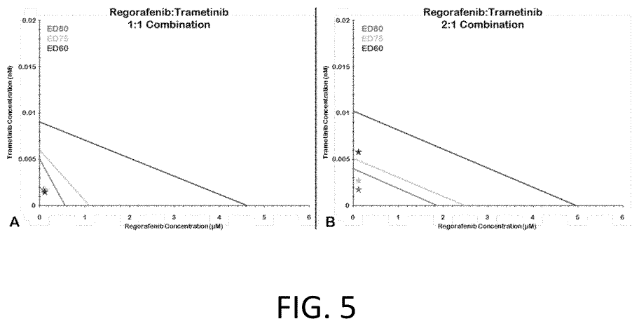

Next, we generated isobolograms and determined the dose requirements for each drug at 20%, 25% and 40% cancer cell death as a read-out for synergy. As shown in FIG. 5, the isobole of the 1:1 and 2:1 regorafenib:trametinib combinations were below the additive isobole for each effect level indicating strong synergy. The isoboles at the 5:1 and 10:1 regorafenib:trametinib combinations were much closer to the additive isoboles for each effect level, indicating slight additive effects for the combinations (data not shown).

### DISCUSSION

More than 80% of pancreatic cancers are ductal adenocarcinomas (PDAC) (47) and as the fourth most common cause of cancer-related death, it is one of the most lethal solid malignancies (48). Although, gemcitabine has been the only validated standard regimen for advanced PDAC for more than a decade, the 5-year survival rate for this disease has not significantly improved over the past 4 decades (38).

Approximately 90% of all PDACs display mutations in the Kirsten rat sarcoma viral oncogene homolog (KRAS), the most frequently mutated oncogene/protein in PDAC (28). Moreover, mutations at codon 12, such as p.G12V (seen in the CAPAN2 cells used in this study), account for ˜98% of all KRAS mutations in PDAC (29). The p.G12V mutation results in constitutive activation of this protein kinase leading to a series of downstream signaling events that mediate uncontrolled increases in cellular proliferation, motility, adhesion, invasion, blocking of apoptosis and resistance to chemotherapy (30). Despite this, no specific RAS inhibitor has been identified and this protein kinases has been widely perceived as “undruggable” (49).

Nevertheless, the development and commercialization of therapeutic agents that, at least indirectly, can block KRAS function through the inhibition of its downstream effectors have been developed. For example, trametinib (MEKINIST®), a selective inhibitor of MEK, is a downstream inhibitor of the MAP kinase signaling pathway constitutively activated by the KRAS p.G12V mutation (49), and it has been demonstrated that such MAP kinase inhibitors are an important therapy for targeting RAS (40).

ABL1, a non-receptor tyrosine kinase, regulates a diverse set of cellular processes controlling cell growth, survival, invasion, adhesion and migration (31). The p.G1060D mutation of ABL1, seen in these CAPAN2 cells, occurs in the actin-binding domain of the kinase and although it has not been functionally characterized, it is believed that this is an activating mutation since it could lead to increases in the cytoplasmic levels of ABL and thus its kinase activity, which is blocked by its binding to the filamentous actin cytoskeleton (33,34). The multi-kinase inhibitor regorafenib, has been shown to target non-receptor tyrosine kinases, including ABL1 (40).

In the present study, we show that both trametinib and regorafenib (individually and in combination) inhibit cell proliferation in CAPAN2 cells bearing activating mutations in KRAS and ABL1. In fact, combinations of these two inhibitors were found to lead to increased cell death at much lower concentrations than either of the drugs in isolation (FIGS. 1-3). To assess the precise type of drug-drug interaction observed, isobolographic analyses were applied. This method allows for the evaluation of the efficaciousness of a combination of active agents regardless of their mechanism of action (38,39). It was found that both a 1:1 combination and a 2:1 combination of regorafenib to trametinib had synergistic effects on cell death at EC80 and EC75, while the 2:1 combination of these two drugs was synergistic for cell death at the EC60 (FIG. 5).

The biphasic response observed when the cells were treated with combination of trametinib and regorafenib, was not seen at any concentration following treatment with either matched monotherapy (FIG. 1-3). This biphasic response is reminiscent of the hormetic effect, which has been described in many human tumor cell lines treated with variety of chemical agents (53). While the exact cause of this effect is unclear, hormesis is thought to be, at least in part, due to the cellular response to stress (53). As described above, it is interesting that treatment with either drug alone did not display such an effect, it appears only when both drugs were used in combination, raising the possibility that the combined inhibition of the kinases at some concentrations is perhaps more stressful for the cell than either drug alone, although this remains to be determined.

Overall the study reports the systematic analysis of monotherapies and combination of anti-cancer drugs matched to genome alterations and support the notion that: 1) matched monotherapies targeting actionable alterations provide significant increases in cell death compared to the standard of care; and 2) more importantly, matched combination therapies have the potential to provide even more effective treatments than either matched monotherapies or the standard of care. When taken together with the recent advances in cancer tumor genomics analysis, and in drug design and development, it is clear that researchers and clinicians now have the opportunity and means to treat cancer as the personal disease that it is.

### OTHER EMBODIMENTS

The detailed description set-forth above is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description which do not depart from the spirit or scope of the present inventive discovery. Such modifications are also intended to fall within the scope of the appended claims.

### REFERENCES CITED

All publications, patents, patent applications and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

Specifically intended to be within the scope of the present invention, and incorporated herein by reference in its entirety, are the following publications:
1. Bennett C W et al. (2016), Cell-free DNA and next-generation sequencing in the service of personalized medicine for lung cancer, Oncotarget 7: 71013-71035.
2. de Bono J S1 et al. (2010), Translating cancer research into targeted therapeutics. Nature 467:543-549.
3. Luthra R (2015) Next-Generation Sequencing in Clinical Molecular Diagnostics of Cancer: Advantages and Challenges, Cancers 7:2023-2036.
4. Meldrum C et al. (2011) Next-Generation Sequencing for Cancer Diagnostics: a Practical Perspective Clin Biochem Rev 32:177-195.
5. Diwakarla C et al. (2017) Advanced pancreatic ductal adenocarcinoma ˜Complexities of treatment and emerging therapeutic options, World J Gastroenterol 23:2276-2285.
6. Pritchard C C et al. (2014) Validation and Implementation of Targeted Capture and Sequencing for the Detection of Actionable Mutation, Copy Number Variation, and Gene Rearrangement in Clinical Cancer Specimens, J Mol Diagnostics 16:57-67.
7. Kou T et al. 2017, Clinical sequencing using a next-generation sequencing-based multiplex gene assay in patients with advanced solid tumors, Cancer Sci April 25. doi: 10.1111/cas.13265. [Epub ahead of print].
8. Osmanbeyoglu H U et al. (2017) Pancancer modelling predicts the context-specific impact of somatic mutations on transcriptional programs, Nat Commun (2017) 8:14249.
9. Shen H et al. (2017) Diagnostic and prognostic value of blood samples for KRAS mutation identification in lung cancer: a meta-analysis, Oncotarget 8:36812-36823.
10. Cicenas J et al. (2017) KRAS, TP53, CDKN2A, SMAD4, BRCA1, and BRCA2 Mutations in Pancreatic Cancer, Cancers 9:42-50.
11. Itamura H et al. (2016) The MEK inhibitor trametinib separates murine graft-versus-host disease from graft-versus-tumor effects, JCI Insight (2016) 1(10):1172.
12. Kang Z et al., The Philadelphia chromosome in leukemogenesis, Chin J Cancer (2016) 35:48
13. Lin X et al. (2017) Regorafenib inhibited gastric cancer cells growth and invasion via CXCR4 activated Wnt pathway PLoS One 10; 12(5)
14. Morgan, M. M., Johnson, B. P., Livingston, M. K., Schuler, L. A., Alarid, E. T., Sung, K. E., and Beebe, D. J. (2016) Personalized in vitro cancer models to predict therapeutic response: Challenges and a framework for improvement. Pharmacol Ther 165, 79-92
15. Martini, M., Vecchione, L., Siena, S., Tejpar, S., and Bardelli, A. (2011) Targeted therapies: how personal should we go? Nat Rev Clin Oncol 9, 87-97
16. Schwaederle, M., Zhao, M., Lee, J. J., Eggermont, A. M., Schilsky, R. L., Mendelsohn, J., Lazar, V., and Kurzrock, R. (2015) Impact of Precision Medicine in Diverse Cancers: A Meta-Analysis of Phase II Clinical Trials. J Clin Oncol 33, 3817-3825
17. Henary, H., Hong, D. S., Falchook, G. S., Tsimberidou, A., George, G. C., Wen, S., Wheler, J., Fu, S., Naing, A., Piha-Paul, S., Janku, F., Kim, K. B., Hwu, P., and Kurzrock, R. (2013) Melanoma patients in a phase I clinic: molecular aberrations, targeted therapy and outcomes. Ann Oncol 24, 2158-2165
18. Von Hoff, D. D., Stephenson, J. J., Jr., Rosen, P., Loesch, D. M., Borad, M. J., Anthony, S., Jameson, G., Brown, S., Cantafio, N., Richards, D. A., Fitch, T. R., Wasserman, E., Fernandez, C., Green, S., Sutherland, W., Bittner, M., Alarcon, A., Mallery, D., and Penny, R. (2010) Pilot study using molecular profiling of patients' tumors to find potential targets and select treatments for their refractory cancers. J Clin Oncol 28, 4877-4883
19. Weiss, G. J., Liang, W. S., Demeure, M. J., Kiefer, J. A., Hostetter, G., Izatt, T., Sinari, S., Christoforides, A., Aldrich, J., Kurdoglu, A., Phillips, L., Benson, H., Reiman, R., Baker, A., Marsh, V., Von Hoff, D. D., Carpten, J. D., and Craig, D. W. (2013) A pilot study using next-generation sequencing in advanced cancers: feasibility and challenges. PLoS One 8, e76438
20. Tsimberidou, A. M., Wen, S., Hong, D. S., Wheler, J. J., Falchook, G. S., Fu, S., Piha-Paul, S., Naing, A., Janku, F., Aldape, K., Ye, Y., Kurzrock, R., and Berry, D. (2014) Personalized medicine for patients with advanced cancer in the phase I program at MD Anderson: validation and landmark analyses. Clin Cancer Res 20, 4827-4836
21. Kass, E. M., Moynahan, M. E., and Jasin, M. (2016) When Genome Maintenance Goes Badly Awry. Mol Cell 62, 777-787
22. Kyriazis, A. A., Kyriazis, A. P., Sternberg, C. N., Sloane, N. H., and Loveless, J. D. (1986) Morphological, biological, biochemical, and karyotypic characteristics of human pancreatic ductal adenocarcinoma Capan-2 in tissue culture and the nude mouse. Cancer Res 46, 5810-5815
23. Thota, R., Pauff, J. M., and Berlin, J. D. (2014) Treatment of metastatic pancreatic adenocarcinoma: a review. Oncology (Williston Park) 28, 70-74
24. Barretina, J., Caponigro, G., Stransky, N., Venkatesan, K., Margolin, A. A., Kim, S., Wilson, C. J., Lehar, J., Kryukov, G. V., Sonkin, D., Reddy, A., Liu, M., Murray, L., Berger, M. F., Monahan, J. E., Morais, P., Meltzer, J., Korejwa, A., Jane-Valbuena, J., Mapa, F. A., Thibault, J., Bric-Furlong, E., Raman, P., Shipway, A., Engels, I. H., Cheng, J., Yu, G. K., Yu, J., Aspesi, P., Jr., de Silva, M., Jagtap, K., Jones, M. D., Wang, L., Hatton, C., Palescandolo, E., Gupta, S., Mahan, S., Sougnez, C., Onofrio, R. C., Liefeld, T., MacConaill, L., Winckler, W., Reich, M., Li, N., Mesirov, J. P., Gabriel, S. B., Getz, G., Ardlie, K., Chan, V., Myer, V. E., Weber, B. L., Porter, J., Warmuth, M., Finan, P., Harris, J. L., Meyerson, M., Golub, T. R., Morrissey, M. P., Sellers, W. R., Schlegel, R., and Garraway, L. A. (2012) The Cancer Cell Line Encyclopedia enables predictive modelling of anticancer drug sensitivity. Nature 483, 603-607
25. Di Veroli, G. Y., Fornari, C., Goldlust, I., Mills, G., Koh, S. B., Bramhall, J. L., Richards, F. M., and Jodrell, D. I. (2015) An automated fitting procedure and software for dose-response curves with multiphasic features. Sci Rep 5, 14701
26. Tallarida, R. J., Porreca, F., and Cowan, A. (1989) Statistical analysis of drug-drug and site-site interactions with isobolograms. Life Sci 45, 947-961
27. van der Heijden, M. S., Yeo, C. J., Hruban, R. H., and Kern, S. E. (2003) Fanconi anemia gene mutations in young-onset pancreatic cancer. Cancer Res 63, 2585-2588
28. Hruban, R. H., Pitman, M. B., and Klimstra, D. S. (2007) Tumors of the pancreas, American Registry of Pathology Series 4.
29. Eser, S., Schnieke, A., Schneider, G., and Saur, D. (2014) Oncogenic KRAS signalling in pancreatic cancer. Br J Cancer 111, 817-822
30. Zavoral, M., Minarikova, P., Zavada, F., Salek, C., and Minarik, M. (2011) Molecular biology of pancreatic cancer. World J Gastroenterol 17, 2897-2908
31. Greuber, E. K., Smith-Pearson, P., Wang, J., and Pendergast, A. M. (2013) Role of ABL family kinases in cancer: from leukaemia to solid tumours. Nat Rev Cancer 13, 559-571
32. Van Etten, R. A. (1999) Cycling, stressed-out and nervous: cellular functions of c-Abl. Trends Cell Biol 9, 179-186
33. de Oliveira, G. A., Rangel, L. P., Costa, D. C., and Silva, J. L. (2015) Misfolding, Aggregation, and Disordered Segments in c-Abl and p53 in Human Cancer. Front Oncol 5, 97
34. Wong, S., and Witte, 0. N. (2004) The BCR-ABL story: bench to bedside and back. Annu Rev Immunol 22, 247-306
35. Lee, B. J., and Shah, N. P. (2017) Identification and characterization of activating ABL1 lb kinase mutations: impact on sensitivity to ATP-competitive and allosteric ABL1 inhibitors. Leukemia (28 Nov. 2016)| doi:10.1038/leu.2016.353.
36. Woodring, P. J., Hunter, T., and Wang, J. Y. (2001) Inhibition of c-Abl tyrosine kinase activity by filamentous actin. J Biol Chem 276, 27104-27110
37. de Sousa Cavalcante, L., and Monteiro, G. (2014) Gemcitabine: metabolism and molecular mechanisms of action, sensitivity and chemoresistance in pancreatic cancer. Eur J Pharmacol 741, 8-16
38. Neuzillet, C., Tijeras-Raballand, A., Bourget, P., Cros, J., Couvelard, A., Sauvanet, A., Vullierme, M. P., Tournigand, C., and Hammel, P. (2015) State of the art and future directions of pancreatic ductal adenocarcinoma therapy. Pharmacol Ther 155, 80-104
39. Bournet, B., Buscail, C., Muscari, F., Cordelier, P., and Buscail, L. (2016) Targeting KRAS for diagnosis, prognosis, and treatment of pancreatic cancer: Hopes and realities. Eur J Cancer 54, 75-83
40. (2012) Application number: 203085Orig1s000, Pharmacology Review(s). in https://www.accessdata.fda.gov/drugsatfda docs/nda/2012/203085Orig1s000PharmR.pdf (Research, C. f. D. E. a. ed.)
41. Franco, J., Witkiewicz, A. K., and Knudsen, E. S. (2014) CDK4/6 inhibitors have potent activity in combination with pathway selective therapeutic agents in models of pancreatic cancer. Oncotarget 5, 6512-6525
42. Deer, E. L., Gonzalez-Hernandez, J., Coursen, J. D., Shea, J. E., Ngatia, J., Scaife, C. L., Firpo, M. A., and Mulvihill, S. J. (2010) Phenotype and genotype of pancreatic cancer cell lines. Pancreas 39, 425-435
43. Chou, T. C. (2010) Drug combination studies and their synergy quantification using the Chou-Talalay method. Cancer Res 70, 440-446
44. Tallarida, R. J. (2001) Drug synergism: its detection and applications. J Pharmacol Exp Ther 298, 865-872
45. Tallarida, R. J. (2006) An overview of drug combination analysis with isobolograms. J Pharmacol Exp Ther 319, 1-7
46. Foucquier, J., and Guedj, M. (2015) Analysis of drug combinations: current methodological landscape. Pharmacol Res Perspect 3, e00149
47. Ruess, D. A., Gorgulu, K., Wormann, S. M., and Algul, H. (2017) Pharmacotherapeutic Management of Pancreatic Ductal Adenocarcinoma: Current and Emerging Concepts. Drugs Aging 34, 331-357
48. Siegel, R. L., Miller, K. D., and Jemal, A. (2016) Cancer statistics, 2016. CA Cancer J Clin 66, 7-30
49. Cox, A. D., Fesik, S. W., Kimmelman, A. C., Luo, J., and Der, C. J. (2014) Drugging the undruggable RAS: Mission possible? Nat Rev Drug Discov 13, 828-851
50. Kiessling, M. K., and Rogler, G. (2015) Targeting the RAS pathway by mitogen-activated protein kinase inhibitors. Swiss Med Wkly 145, w14207
51. Teuschler, L., Klaunig, J., Carney, E., Chambers, J., Conolly, R., Gennings, C., Giesy, J., Hertzberg, R., Klaassen, C., Kodell, R., Paustenbach, D., and Yang, R. (2002) Support of science-based decisions concerning the evaluation of the toxicology of mixtures: a new beginning. Regul Toxicol Pharmacol 36, 34-39
52. Gumbarewicz, E., Luszczki, J. J., Wawruszak, A., Dmoszynska-Graniczka, M., Grabarska, A. J., Jarzab, A. M., Polberg, K., and Stepulak, A. (2016) Isobolographic analysis demonstrates additive effect of cisplatin and HDIs combined treatment augmenting their anti-cancer activity in lung cancer cell lines. Am J Cancer Res 6, 2831-2845.
53. Calabrese, E. J. (2005) Cancer biology and hormesis: human tumor cell lines commonly display hormetic (biphasic) dose responses. Crit Rev Toxicol 35, 463-582.

What is claimed is:

1. A method for treating a cancer in a patient in need thereof, the method comprising: (a) identifying two or more target genes in the cancer cells each of which has a pathogenic and actionable mutation; (b) using a list of gene-drug substances interactions, and (c) administering to the patient one or more substances targeting the actionable mutation for each of the two or more target genes identified in (a).

2. The method of claim 1, wherein the identifying comprises use of NGS sequencing.

3. The method of claim 1, wherein the combination therapy has a synergistic therapeutic effect.

4. The method of claim 1, wherein the cancer is selected from the group consisting of solid tumors and non-solid tumors.

5. The method of claim 1, wherein the sample comprises cancer cells or a cell free sample comprising cancer DNA.

6. The method of claim 1, wherein each of the two or more target genes map to different pathways.

7. The method of claim 1, wherein the cancer is pancreatic ductal adenocarcinoma and the two or more target genes are KRAS and ABL1.

8. The method of claim 7, wherein KRAS contains a pathogenic alteration.

9. The method of claim 7, wherein ABL1 contains a pathogenic alteration.

10. The method of claim 7, wherein both KRAS and ABL1 contain a pathogenic alteration.

11. The method of claim 7, wherein ABL1 is FANCC ABL1.

12. The method of claim 7, wherein G12 of KRAS is substituted with a non-standard amino acid selected from the group consisting of A, C, D, E, F, H, I, K, L, M, N, P, Q, R, S, T, V, and W.

13. The method of claim 7, wherein G1060 of ABL1 is substituted with a non-standard amino acid selected from the group consisting of A, C, D, E, F, H, I, K, L, M, N, P, Q, R, S, T, V, and W.

14. The method of claim 7, wherein:
G12 of KRAS is substituted with a non-standard amino acid selected from the group consisting of A, C, D, E, F, H, I, K, L, M, N, P, Q, R, S, T, V, and W; and/or
G1060 of ABL1 is substituted with a non-standard amino acid selected from the group consisting of A, C, D, E, F, H, I, K, L, M, N, P, Q, R, S, T, V, and W.

15. The method of claim 7, wherein:
G12 of KRAS is substituted with a non-standard amino acid selected from the group consisting of A, C, D, E, F, H, I, K, L, M, N, P, Q, R, S, T, V, and W; and/or
G1060 of ABL1 is substituted with a non-standard amino acid selected from the group consisting of A, C, D, E, F, H, I, K, L, M, N, P, Q, R, S, T, V, and W; and/or
E521 of FANCC is substituted with a non-standard amino acid selected from the group consisting of A, C, D, E, F, H, I, K, L, M, N, P, Q, R, S, T, V, and W.

16. The method of claim 7, wherein the one or more substances targeting the KRAS gene is an inhibitor of mitogen/extracellular signal-related kinase (MEK).

17. The method of claim 16, wherein the MEK inhibitor is trametinib.

18. The method of claim 7, wherein the one or more substances targeting the ABL1 gene is a tyrosine kinase inhibitor.

19. The method of claim 18, wherein the tyrosine kinase inhibitor is regorafenib.

20. The method of claim 1, wherein a combination therapy of trametinib and regorafenib is administered to the patient.

21. The method of claim 20, wherein the trametinib and at least one additional anti-cancer agent are administered sequentially.

22. The method of claim 20, wherein the trametinib and at least one additional anti-cancer agent are administered concurrently.

23. The method of claim 20, wherein the subject is human.

24. The method of claim 20, wherein the regorafenib and at least one additional anti-cancer agent are administered sequentially.

25. The method of claim 20, wherein the regorafenib and at least one additional anti-cancer agent are administered concurrently.

26. A method for inhibiting cancer cell proliferation, comprising exposing cancer cells to trametinib in combination with at least one additional anti-cancer agent, wherein the combination provides an enhanced anti-cancerous effect compared to the effect of trametinib alone and/or at least one additional anti-cancer agent administered alone.

27. A method for identifying a treatment for a patient having cancer, the method comprising (a) obtaining from the patient, a sample containing cancer cells; (b) obtaining the results of NGS sequencing of this sample; (c) identifying two or more target genes in the cancer cells each of which has an actionable mutation, (d) culturing the cancer cells in presence of one or more substances targeting the actionable mutation for each of the two or more identified target genes identified in (c), (e) measuring cancer cell viability in presence of the one or more substances and (f) concluding that the treatment might be effective in the patient if the viability of the cells in the presence of the one or more substance is (i) less than the viability in absence of the two or more substances; (ii) less than the viability in the presence of one or more standard-of-care, non-targeted, substance; (iii) less than the viability in the presence of a targeted but non-matched substance (=negative controls).

28. The method of claim 27, wherein each of the two or more identified target genes maps to a different pathway.

29. The method of claim 27, wherein the cancer is pancreatic ductal adenocarcinoma and the two or more identified target genes are KRAS and ABL1.

30. The method of claim 29, wherein KRAS contains a pathogenic alteration.12B. The method of claim 12, wherein ABL1 contains a pathogenic alteration.12C. The method of claim 12, wherein both KRAS and ABL1 contain a pathogenic alteration

31. The method of claim 29, wherein KRAS, ABL1, and FANCC contain a pathogenic alteration

32. The method of claim 29, wherein G12 of KRAS is substituted with a non-standard amino acid selected from the group consisting of A, C, D, E, F, H, I, K, L, M, N, P, Q, R, S, T, V, and W.

33. The method of claim 12, wherein said wherein G1060 of ABL1 is substituted with a non-standard amino acid selected from the group consisting of A, C, D, E, F, H, I, K, L, M, N, P, Q, R, S, T, V, and W.

34. The method of claim 29, wherein:
G12 of KRAS is substituted with a non-standard amino acid selected from the group consisting of A, C, D, E, F, H, I, K, L, M, N, P, Q, R, S, T, V, and W; and/or
G1060 of ABL1 is substituted with a non-standard amino acid selected from the group consisting of A, C, D, E, F, H, I, K, L, M, N, P, Q, R, S, T, V, and W.

35. The method of claim 29, wherein:
G12 of KRAS is substituted with a non-standard amino acid selected from the group consisting of A, C, D, E, F, H, I, K, L, M, N, P, Q, R, S, T, V, and W; and/or
G1060 of ABL1 is substituted with a non-standard amino acid selected from the group consisting of A, C, D, E, F, H, I, K, L, M, N, P, Q, R, S, T, V, and W; and/or
E521 of FANCC is substituted with a non-standard amino acid selected from the group consisting of A, C, D, E, F, H, I, K, L, M, N, P, Q, R, S, T, V, and W.

36. The method of claim 29, wherein the one or more substances targeting the KRAS gene is an inhibitor of mitogen/extracellular signal-related kinase (MEK).

37. The method of claim 36, wherein the MEK inhibitor is trametinib.

38. The method of claim 29, wherein the one or more substances targeting the ABL1 gene is a tyrosine kinase inhibitor.

39. The method of claim 38, wherein the tyrosine kinase inhibitor is regorafenib.

40. The method of claim 10, wherein a combination therapy of trametinib and regorafenib is identified as a treatment method for the patient having cancer.

41. A composition for treating cancer, the composition comprising a first component consisting of an effective amount of trametinib and second component comprising an effective amount of at least one additional anti-cancer agent.

42. The composition of claim 41, wherein the collective amount of trametinib and at least one additional anti-cancer agent provides a synergistic therapeutic anti-cancer effect.

43. The composition of claim 41, wherein the collective amount of trametinib and at least one additional anti-cancer agent provides an enhanced therapeutic anti-cancer effect.

44. The composition of claim 41, wherein the collective amount of trametinib and at least one additional anti-cancer agent provides a synergistic therapeutic anti-cancer effect.

45. The composition of claim 41, wherein the cancer is selected from the group consisting of solid tumor and non-solid tumors.

46. The composition of claim 41, wherein the cancer is a solid tumor selected from the group consisting of colorectal cancer, gastric cancer, colorectal cancer, pancreatic cancer and prostate cancer.

47. The composition of claim 41, wherein the at least one additional anti-cancer agent is a chemotherapeutic agent.

48. A composition for treating cancer, the composition comprising a first component consisting of an effective amount of regorafenib and second component comprising an effective amount of at least one additional anti-cancer agent.

49. The composition of claim 48, wherein the collective amount of regorafenib and at least one additional anti-cancer agent provides an enhanced therapeutic anti-cancer effect.

50. The composition of claim 48, wherein the collective amount of regorafenib and at least one additional anti-cancer agent provides a synergistic therapeutic anti-cancer effect.

51. The composition of claim 48, wherein the cancer is selected from the group consisting of solid tumor and non-solid tumors.

52. A kit for identifying a treatment method for a patient having cancer, the kit comprising (a) data of two or more target genes in the cancer cells (using NGS sequencing or other methods) each of which has a pathogenic and actionable mutation; (b) list of gene-drug substances interactions, and (c) one or more substances targeting the actionable mutation for each of the two or more identified target genes identified in (a), and (d) medium for culturing cancer cells from the patient in presence of each of the one or more substances in (c), packaged in one or more containers.

53. The kit of claim 52, wherein the cancer is pancreatic ductal adenocarcinoma and the two or more identified target genes are KRAS and ABL1.

54. The kit of claim 53, wherein the one or more substances targeting the KRAS gene is an inhibitor of mitogen/extracellular signal-related kinase (MEK).

55. The kit of claim 54, wherein the MEK inhibitor is trametinib.

56. The kit of claim 53, wherein the one or more substances targeting the ABL1 gene is a tyrosine kinase inhibitor.

57. The kit of claim 56, wherein the tyrosine kinase inhibitor is regorafenib.
